(19) United States
(12) Patent Application Publication
(10) Pub. No.: US 2004/0039440 A1
(43) Pub. Date: Feb. 26, 2004

(54) Biodegradable sleeves for intravascular devices

(76) Inventor: David R. Elmaleh, Newton, MA (US)

Correspondence Address: GREENBURG TRAURIG, LLP, ONE INTERNATIONAL PLACE, 3RD FLOOR, BOSTON, MA, 02110, US

(21) Appl. No.: 10/208,950

(22) Filed: Jul. 31, 2002

(51) Int. Cl. A61F 2/06

(52) U.S. Cl. 623/1.42; 427/2.1

(57) ABSTRACT

An intravascular device having a removable biocompatible sleeve capable of being pulled substantially over the length of the device is provided. The device may be a substantially expandable tubular body defined by a mesh framework. The sleeve can include a plurality of perforations to permit fluid communication with the mesh framework. The sleeve may be biodegradable and may contain a pharmacotherapeutic agent to permit treatment of certain conditions. After deployment to a site of interest, the device and more specifically, the sleeve, can provide local delivery of sustained or controlled therapeutic dose of the suitable pharmacotherapeutic agent.

### FIELD OF THE INVENTION

The present invention relates to intravascular devices, and more particularly, to intravascular devices having a biodegradable sleeve thereon.

### BACKGROUND OF THE INVENTION

Many medical intravascular devices are currently being used either temporarily or permanently inside the human body. One example of an intravascular device includes a stent for use in, for instance, coronary angioplasty. Stents are small mechanical devices, which can be implanted into, for example, a blood vessel to hold open and support the constricted vessel, so as to prevent a re-narrowing or closure of the vessel subsequent to an angioplasty procedure. Stent generally designed to include small metal scaffolds comprising a mesh or perforated tube, for direct insertion to the site of closure or narrowing, and can be mechanically expanded by, for instance, a balloon to reopen the vessel at the site of closure. For proper positioning, stents may be made to permit visualization during and after deployment using imaging techniques such as x-ray radiography and x-ray fluoroscopy. However, due to the nature of the materials used to construct these intravascular devices and their small size, visualization of these devices can often be poor or non-existent.

The mechanical reopening of a constricted vessel with a balloon can sometimes lead to balloon-related injuries of the tissues at the site of closure. Such injuries can often stimulate tissue proliferation at the reopened site during the healing process, and which proliferation can result in pronounced neointimal hyperplasia or restenosis. Restenosis remains the most common post-stenting clinical problem, and requires effective intervention or counter-measures to prevent and/or control its reoccurrence.

Currently, methods for preventing or controlling restenosis are specifically aimed at influencing factors believed to be involved in the body's response to external or internal tissue stimulants, such as angioplasty, stenting procedures, and/or viruses. Common countermeasures which have been used to prevent or control restenosis generally fall into the one of several categories, including (1) mechanical atheroablative techniques, such as debulking, vascular filters, and emboli-trapping devices, (2) ultrasound-initiated atheroablative techniques, (3) light-assisted procedures, predominantly excimer laser angioplasty, (4) pharmacological agents and gene therapy, (5) ultraviolet photophoresis, believed to be an immune modulator, (6) radiation therapy, such as external and endovascular brachytherapy, and (7) re-stenting.

In addition, modifications to stent designs and materials have been proposed to prevent and/or control restenosis. In one approach, non-metallic, biodegradable stent materials, such as high molecular weight Poly-1-lactic acid (PLLA) is used.

Numerous inorganic coatings and surface treatments have also been developed to improve chemical inertness and biocompatibility of metallic stents. Some coatings, such as gold, however, yield a higher rate of in-stent restenosis than uncoated stents. Others, including silicon carbide and turbostatic carbon, show promise but additional studies must be done.

Organic coatings, including both synthetic and natural coatings, have also been widely studied. Among the synthetic coatings studied are Dacron, polyester, polyurethane, polytetrafluoroethylene (PTFE), polyethylacrylate/polymethylmetahcrylate, polyvinyl chloride, silicone, collagen, and iridium oxide. Results of studies, such as those with PTFE-coated stents, are disappointing or mixed at best, as there are high occurrences of late thrombo-occlusive events. With only a very few exceptions, the general consensus is that any favorable outcome was not associated with treatment of conventional in-stent restenosis using PTFE-coated stents.

Intracoronary intervention have also been employed to reduce neointima formation by reducing smooth muscle cell proliferation after balloon angioplasty. However, such intervention is often complicated by subacute and late thrombosis. Coronary thrombo-aspiration and coronary pulsed-spray procedures, followed by immediate endovascular therapy, have also been particularly helpful in removing thrombotic material associated with plaque.

In addition, pharmacotherapeutic agents have been used for the treatment of some of the major post-angioplasty complications, including immunosuppresants, anticoagulants and anti-inflammatory compounds, chemotherapy agents, antibiotics, antiallergenic drugs, cell cycle inhibitors, gene therapy compounds, and ceramide therapy compounds.

Pharmacotherapeutic agents can be delivered either systemically or locally. Systemic treatment has shown limited success in reducing restenosis following stent implantation, a result believed to be due to inadequate concentration of the pharmacotherapeutic agents at the site of injury. Increased dose administration, however, is constrained by possible systemic toxicity. It has been observed that local delivery of higher doses via drug eluting stents can significantly reduce adverse systemic effects.

Gene therapy have also been employed in the treatment of restenosis. The procedure is directed towards smooth muscle cells and involves gene transfer via DNA, with or without integration of chromosomes, into selected cells. In transduction without integration, the gene is delivered to both cytoplasm and nucleus and is therefore non-selective. Gene transfer for integration employs retrovirus to affect growth stimulators.

Antibiotics, likewise, has been used in the treatment of coronary artery disease. It is known that antibiotics are effective in controlling inflammation caused by a variety of infectious agents found in fatty plaques blocking the arteries. Results of clinical investigation, such as with azithromycin, suggest a modest antibiotic benefits for heart patients.

Similarly, a phospholipid exhibiting immunosuppressive properties, has been shown to block T-cell activation and proliferation, inhibit Taxol-induced cell cycle apoptosis, and activate protein kinase signal translation in malignant myogenic cells. Rapamycin and its analogs exhibit anti-tumor activities at relatively low dose levels, while inducing only mild side effects, an extremely important aspect of patient care.

### SUMMARY OF THE INVENTION

The present invention provides, in one embodiment, an intravascular device, such as a stent, for maintaining an opening within a constricted vessel. The device may also be used for local delivery of at least one pharmacotherapeutic agent to an intravascular site, for the treatment of, for instance, restenosis following, for example, balloon angioplasty.

The intravascular device, in accordance with an embodiment of the invention, includes an expandable substantially tubular body defined by a mesh framework, a removable biocompatible sleeve for pulling over an entire length of the framework. In an embodiment, the sleeve may be extensible to permit its expansion with the tubular body. The device further includes a plurality of perforations throughout the sleeve to permit communication with the mesh framework. If desired, the sleeve may include a pharmacotherapeutic agent for the treatment or prevention of certain conditions, and may be made from a biodegradable polymer.

In an alternate embodiment, the intravascular device includes an expandable substantially tubular frame defined by a plurality of overlaying filaments. The device also includes a plurality of openings between the overlaying filaments. In this embodiment, however, the device is provided with a biocompatible extensible sleeve positioned about each filament, and can be expanded with the tubular frame.

The device of the present invention may be manufactured by initially providing an expandable substantially tubular body defined by a mesh framework. Thereafter, a biocompatible extensible sleeve having opposite open ends, a body portion extending between the open ends, and a plurality of perforations on the body may be expanded at one of the open ends of the sleeve along its diameter. Next one end of the tubular body may be placed within the expanded open end of the sleeve. Once therein, the sleeve may be pulled over the remainder of the tubular body until the sleeve substantially covers the body.

In another embodiment, the device may be manufactured by providing an expandable tubular frame defined by a plurality of overlaying filaments. Next a biocompatible polymeric solution may be formed and subsequently deposited on to each of the filaments of the tubular frame, so as to encapsulate each of the filaments. Thereafter, the solution is permitted to dry to form an extensible sleeve around each filament.

The present invention further provides a kit having, in one embodiment, a removable biocompatible sleeve for positioning over a mesh framework of a substantially tubular intravascular device. The sleeve, in an embodiment, is designed to be extensible to permit its expansion with the mesh framework, and includes a plurality of perforations to permit communication with the mesh framework.

Alternative, a kit can be provided to include a polymeric mixture for forming a biocompatible polymeric solution. The kit can also include a mechanism for applying the solution on to filaments of a tubular intravascular device, so as to encapsulate each of the filaments.

### BRIEF DESCRIPTION OF THE DRAWINGS

FIG. 1 illustrates a sleeved stent for use in accordance with an embodiment of the present invention.

FIG. 2 illustrates a mesh framework defining a stent for use in accordance with an embodiment of the invention.

FIG. 3 illustrates an extensible sleeve for use with the framework shown in FIG. 2.

FIG. 4 illustrates a stent with an extensible sleeve around each of the filament of a stent, as set forth in another embodiment of the present invention.

FIG. 5 illustrates a longitudinal view of a sleeve and filament shown in FIG. 4

### DETAILED DESCRIPTION OF THE SPECIFIC EMBODIMENTS

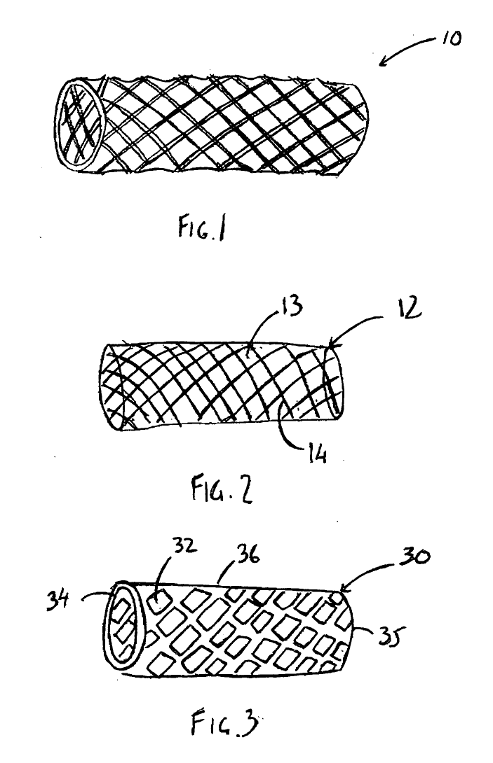

As illustrated in FIG. 1, there is provided, in accordance with an embodiment of the present invention, an intravascular device, such as sleeved stent 10, for maintaining an open lumen in a vascular structure, such as a blood vessel or an artery, and for locally delivering drug to a tissue-injured site caused by, for instance, angioplasty, where over a period of time a therapeutic dose of drug(s) may be released for the treatment of, for example, restenosis.

Previously, local drug delivery to post-angioplasty sites has been accomplished directly from an endovascular catheter. Delivery via an endovascular catheter normally involves delivering a large dose of drug in a very short time period. Because maximum benefits can be achieved by sustained drug delivery, delivery of a large dose in a short time period may not be optimal in many instances.

The stent 10 of the present invention, as shown in FIG. 2, includes, in one embodiment, a substantially tubular mesh framework 12 having openings 13 defined by filaments 14. As the stent 10 will be used to support an opening at a site which was previously closed to maintain a passage therethrough, the mesh framework 12 of stent 10 needs to be made from a material that is sufficiently strong to maintain and support the opening. Although shown in an unexpanded state, the stent 10 can be expanded when positioned at the site of interest. Accordingly, the material from which the framework 12 is made also needs to be sufficiently pliable. In one embodiment of the invention, a material from which the mesh framework 12 may be made includes metal. By providing the stent 10 with a metallic framework 12, the stent 10 may also be visualized, for example, by flouroscopy during placement of the stent 10 within a vessel.

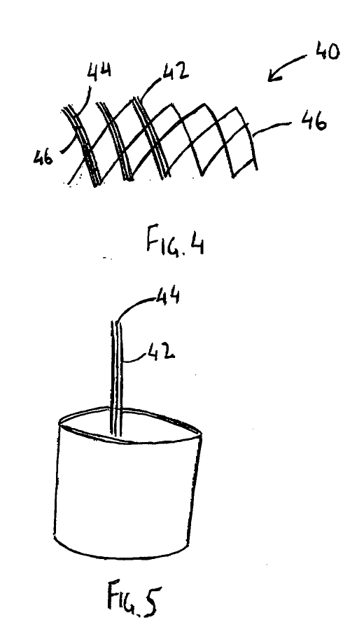

The stent 10 further includes a sleeve 30, as shown in FIG. 3, which can be pulled over an entire length of the stent 10. The sleeve 30, in one embodiment, includes opposite open ends 34 and 35, a body portion 36 extending between the open ends, and a plurality of perforations 32 on the body portion 36. The sleeve 30 can be designed so that the body portion 36 extends over the openings 13 and filaments 14 of the entire mesh framework 12 to substantially covered the stent 10. The perforations 32 on the body portion 36 of sleeve 30, in one embodiment, can be designed so that they substantially compliment the openings 13 of stent 10. In this manner, the sleeve 30 may be permeable to, for instance, blood components, and permits communication within the framework 12 if needed. It is believed that presence of perforations 32 can provide proper tissue (e.g., endothial cell) growth at, for example, a post-angioplasty stented site. In addition, the perforations 32 may provide an space through which surrounding tissue may extend therethrough to secure the stent 10 in place. Perforations 32 on the body portion 36 of sleeve 30 may be created by conventional means, including methods involving the use of lasers, mechanical puncturing, or etching.

The sleeve 30, in accordance with an embodiment, may be made from a biocompatible material, so as to minimize toxic reactions from surrounding tissues. Moreover, as the stent 30 will be expanded once placed at the site of constriction, the sleeve 30 can be provided with the ability to be extensible, so as to permit its expansion with the mesh framework 12.

The sleeve 30 may also serve as a storage and direct transport vehicle for the local delivery of, for instance, restenosis-inhibiting pharmaceuticals. For use as a drug-eluting vehicle, the covering sleeve 30 may include a uniform thickness throughout its entire length and may be made from a biodegradable biocompatible polymer. Such a polymer, in one embodiment, may be poly(glycerol-sebacate) or PGS, similar to that described in Wang et al., A tough biodegradable elastomer, Nature 20(6): pp. 602-606 (June 2002), which is hereby incorporated herein by reference. The sleeve 30, according to an embodiment of the present invention, can include at least one of the pharmacotherapeutic agents mentioned above incorporated throughout the sleeve 30 for subsequent local delivery. Alternatively, the pharmacotherapeutic agent or agents can be positioned within each layer of a multiple layer sleeve 30.

Examples of pharmacotherapeutic agents which may be incorporated within the sleeve include Rapamycin, a phospholipid exhibiting immunosuppressive properties. Heparin and glycosaminoglycans are anticoagulants which may be delivered locally after stent implantation. These anticoagulants interact with growth factors and other glycoproteins, which may reduce neointimal proliferation.

Abciximab is a genetically engineered fragment of a chimeric human-murine monoclonal antibody. It is a glycoprotein inhibitor and works by inhibiting the binding of fibrinogen and other substances to glycoprotein receptor (GBIIb/IIIa) on blood platelets integral to aggregation and clotting. Abciximab appears to be effective in preventing platelet aggregation when used with aspirin and heparin, and appears to be effective in preventing abrupt closure of arteries.

Antibiotics, likewise, can be used in the treatment of coronary artery disease. It is known that antibiotics are effective in controlling inflammation caused by a variety of infectious agents found in fatty plaques blocking the arteries. Azithromycin has been observed to provide modest antibiotic benefits for heart patients.

Other pharmacotherapeutic agents which can be incorporated into the sleeve 30 includes radionuclides for use in the treatment of diseased tissues, and enzymes, which may be encapsulated within a carrier, for instance, a biodegradable sol-gel capsule embedded within the sleeve 30.

It should be appreciated that the concentration of pharmacotherapeutic agent or agents, as well as the rate of degradation of the sleeve 30, can be adjusted according to the treatment for which the stent 10 is being used, so that the rate of release of the agent or agents would be appropriate and sufficient for the treatment.

The sleeved stent 10 of the present invention may be manufactured by initially providing an expandable substantially tubular mesh framework 12. Such a mesh framework 12 can be specially manufactured or commercially obtained from any known source. Thereafter, the biocompatible extensible sleeve 30 may be positioned adjacent the framework 12 and expanded at one of the open ends 34 or 35. Next one end of the tubular framework 12 may be placed within the expanded open end 34 or 35 of the sleeve 30. Once therein, the sleeve 30 may be pulled over the remainder of the periphery of the tubular framework 12 until the sleeve 12 substantially covers the framework 12.

It should be noted that the sleeve 30 may be made to include an outer wall and an inner wall separated by a space (not shown), similar to a glove. In such an embodiment, one end of the framework 12 can be placed within the space between the walls of the sleeve 30 and outer and inner walls of the sleeve 30 can be pulled over both an outer surface of the framework 12 and an inner surface of the framework 12 to completely cover the framework.

Looking now at FIG. 4, there is illustrated a sleeved stent 40, in accordance with another embodiment of the present invention. Stent 40 is similar to stent 10 described above. However, instead of having a covering sleeve 30 extending over the entire length of the mesh framework 12, stent 40 is providing with a sleeve 42 over each of filaments 44 comprising framework 46. The sleeve 42 may be made from the same polymer as noted above and may include at least one pharmacotherapeutic agent as set forth above.

The covering of each filament 44 by a sleeve 42 may be accomplished using methods known in the art. For example, the framework 46 of stent 40 may be immersed into a polymeric mixture, so as to permit the mixture to be deposited on to each of the filaments 44. The mixture deposited on each filament 44 may subsequently be dried to generate a sleeve 42 thereabout. Alternatively, as shown in FIG. 5, a plurality of individual filaments 44 may be immersed into a polymeric mixture, to deposit the mixture thereon. After the mixture is permitted to dry into a sleeve about each filaments 44, the filaments may be attached to one another to form framework 46, and thus sleeved stent 40. In another embodiment, individual sleeves 42 may also be used to cover each filament 44, which may thereafter by connected to form framework 46.

To enhance the placement of the sleeve 42 about each filament, the sleeve 42 may be attached to each filament 44, for instance, by adhesion. Examples of adhesion protocols which may be used in connection with the present invention includes gluing or by derivatizing the metal filaments along the entire length of the filament 44 or, as shown in FIG. 4, in spaced locations 46.

The present invention further provides a kit having, in one embodiment, a removable biocompatible sleeve 30 for positioning over a mesh framework of a substantially tubular intravascular device. The sleeve 30, in an embodiment, is designed to be extensible to permit its expansion with the mesh framework, and includes a plurality of perforations to permit communication with the mesh framework. The sleeve 30 can be made to include at least one pharmacotherapeutic agent.

Alternative, a kit can be provided to include a polymeric mixture for forming a biocompatible polymeric solution. The kit can also include a mechanism for applying the solution on to a framework and/or filaments of a tubular intravascular device, so as to cover each of the filaments.

The sleeved stent of the present invention may be used to maintain an opening within a variety of different vessels. For instance, the sleeved stent may be placed within a coronary artery or a carotid artery. The sleeved stent may also be used to constrict a passageway, for instance, the coronary sinus, among others. To constrict a passageway, the sleeve on the stent may be made so that it is substantially resistant to expansion, so as to permit the sleeve to exert a force on the tubular framework to constrict the tubular framework. The sleeved stent may also be used as a renal stent, gastrointestinal stent, radiation and chemotherapy stent.

While the invention has been described in connection with the specific embodiments thereof, it will be understood that it is capable of further modification. For instance, the sleeve 30 may be used to cover non-metallic stents and may be made with multiple layers, each with a different rate of degradation. The sleeve 30 may also be adapted for use with other intravascular devices for implantation within a patient's body. Furthermore, this application is intended to cover any variations, uses, or adaptations of the invention, including such departures from the present disclosure as come within known or customary practice in the art to which the invention pertains, and as fall within the scope of the appended claims.

What is claimed is:

1. An intravascular device comprising:
an expandable substantially tubular body defined by a mesh framework;
a removable biocompatible sleeve for pulling over the mesh framework, the sleeve being extensible to permit expansion with the body; and
a plurality of perforations positioned on the sleeve to permit communication with the mesh framework.

2. A device as set forth in claim 1, wherein the mesh framework includes openings between filaments defining the framework which substantially compliments the perforations on the sleeve.

3. A device as set forth in claim 1, wherein the body is metallic.

4. A device as set forth in claim 1, wherein the biocompatible sleeve includes a substantially uniform thickness along the entire sleeve.

5. A device as set forth in claim 1, wherein the biocompatible sleeve is made from a polymer.

6. A device as set forth in claim 5, wherein the polymer is biodegrable.

7. A device as set forth in claim 5, wherein the polymer is poly(glycerol-sebacate) (PGS).

8. A device as set forth in claim 1, wherein the biocompatible sleeve includes a pharmacotherapeutic agent.

9. A device as set forth in claim 8, wherein the pharmacotherapeutic agent includes at least one of an immunosuppressant, an antibiotic, a cell cycle inhibitor, an antiinflammatory, an anticoagulant, an antiallergen, and a gene therapy and a ceramide therapy compound.

10. A device as set forth in claim 7, wherein the pharmacotherapeutic agent is encapsulated within a carrier mixed throughout the sleeve.

11. A method of manufacturing an intravascular device, the method comprising:
providing an expandable substantially tubular body defined by a mesh framework;
providing a biocompatible extensible sleeve having opposite open ends, a body portion extending between the open ends, and a plurality of perforation on the body;
expanding at least one open end of the sleeve along its diameter;
placing one end of the tubular body within the expanded open end of the sleeve; and
pulling the sleeve over the remainder of the tubular body until the sleeve substantially covers the tubular body.

12. An intravascular device comprising:
an expandable substantially tubular frame defined by a plurality of overlaying filaments;
a plurality of openings between overlaying filaments; and
a biocompatible sleeve positioned about each filament, the sleeve being extensible to permit expansion with the tubular frame.

13. A device as set forth in claim 12, wherein the biocompatible sleeve is made from a polymer.

14. A device as set forth in claim 13, wherein the polymer is biodegrable.

15. A device as set forth in claim 13, wherein the polymer is poly(glycerol-sebacate) (PGS).

16. A device as set forth in claim 12, wherein the biocompatible sleeve includes a pharmacotherapeutic agent.

17. A device as set forth in claim 16, wherein the pharmacotherapeutic agent is encapsulated within a carrier mixed throughout the sleeve.

18. A method of manufacturing an intravascular device for local delivery of a pharmacotherapeutic agent, the method comprising:
providing an expandable substantially tubular frame defined by a plurality of overlaying filaments
forming a polymeric solution;
applying the solution on to the filaments of the tubular frame, so as to encapsulate the filament; and
permitting an extensible sleeve to form from the solution applied to the filaments.

19. A method as set forth in claim 18, wherein the step of forming includes adding at least one pharmacotherapeutic agent into the polymeric solution, so as to generate a polymer-agent mixture.

20. A method as set forth in claim 18, wherein the step of applying includes dipping the tubular frame into the polymeric solution to permit the solution to cover each of the filaments.

21. A method as set forth in claim 18, wherein the step of applying includes wrapping a sheet made from the polymeric solution around each filament, so as to form a sleeve therearound.

22. A method as set forth in claim 21, wherein the step of wrapping includes gluing each sleeve to each filament.

23 A method as set forth in claim 21, wherein the step of wrapping includes derivatizing each filament to permit attachment of each sleeve thereto.

24. A kit comprising:
a removable biocompatible sleeve for positioning over the mesh framework of a substantially tubular intravascular device,
the sleeve being extensible to permit expansion with the mesh framework, and including a plurality of perforation to permit communication with the mesh framework.

25. A kit comprising:
a polymeric mixture for forming a polymeric solution;
a mechanism for applying the solution on to filaments of a tubular intravascular device, so as to encapsulate the filament.
